(19) United States
(12) Patent Application Publication
(10) Pub. No.: US 2026/0355466 A1
(43) Pub. Date: Nov. 12, 2026

(54) STEERABLE CATHETER

(71) Applicant: BOSTON SCIENTIFIC SCIMED, INC., Maple Grove, MN (US)

(72) Inventors: Sarah Tian-Hong Hua, Plymouth, MN (US); Samuel Shreve, Minneapolis, MN (US)

(73) Assignee: BOSTON SCIENTIFIC SCIMED, INC., MAPLE GROVE, MN (US)

(21) Appl. No.: 19/673,271

(22) Filed: May 11, 2026

Related U.S. Application Data
(60) Provisional application No. 63/804,225, filed on May 12, 2025.

(51) Int. Cl. A61M 25/01; A61M 25/00

(52) U.S. Cl. CPC A61M 25/0136; A61M 25/0097; A61M 2205/582; A61M 2205/583; A61M 2205/586

(57) ABSTRACT

A steerable catheter includes an elongate sheath extending distally from a handle assembly including a handle shell including a hand grip portion, a steering mechanism including a knob that is rotatable relative to the handle shell, and a longitudinal extension disposed opposite the hand grip portion relative to the knob. The steering mechanism is configured to deflect a distal portion of the elongate sheath in at least one direction. The handle assembly may include a second longitudinal extension disposed opposite the hand grip portion relative to the knob, wherein the second longitudinal extension is disposed opposite the longitudinal extension relative to a central longitudinal axis of the handle assembly. The handle assembly may be configured to permit concomitant medical procedures requiring both left-handed steering of the elongate sheath and right-handed steering of the elongate sheath.

### CROSS-REFERENCE TO RELATED APPLICATIONS

This application claims the benefit of priority of U.S. Provisional Application No. 63/804,225 filed May 12, 2025, the entire disclosure of which is hereby incorporated by reference.

### TECHNICAL FIELD

The disclosure relates generally to medical devices and more particularly to steerable catheters, sheaths, and/or elongate tubular shafts.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In one example, a steerable catheter may comprise an elongate sheath extending distally from a handle assembly. The handle assembly may comprise a handle shell including a hand grip portion, a steering mechanism including a knob that is rotatable relative to the handle shell, and a longitudinal extension disposed opposite the hand grip portion relative to the knob. The steering mechanism may be configured to deflect a distal portion of the elongate sheath in at least one direction.

In addition, or alternatively to any example disclosed herein, the longitudinal extension is offset from a central longitudinal axis of the handle assembly.

In addition, or alternatively to any example disclosed herein, the handle assembly comprises a proximal hub structure comprising a proximal hub disposed proximal of the handle shell and a tubular shaft extending distally from the proximal hub within the handle shell.

In addition, or alternatively to any example disclosed herein, the longitudinal extension is coupled directly to the proximal hub.

In addition, or alternatively to any example disclosed herein, the longitudinal extension is monolithically formed with the proximal hub.

In addition, or alternatively to any example disclosed herein, the longitudinal extension is detachable from the proximal hub.

In addition, or alternatively to any example disclosed herein, the proximal hub comprises a receptacle that opens radially outward in a proximal direction and the longitudinal extension comprises a projection configured to matingly engage with the receptacle.

In addition, or alternatively to any example disclosed herein, the longitudinal extension comprises a lumen extending longitudinally therein.

In addition, or alternatively to any example disclosed herein, the lumen of the longitudinal extension opens toward a central longitudinal axis of the handle assembly proximate a side port of the proximal hub.

In addition, or alternatively to any example disclosed herein, the knob is rotatable by a first hand of a user disposed on the hand grip portion of the handle shell and by a second hand of the user disposed on the longitudinal extension.

In addition, or alternatively to any example disclosed herein, the handle assembly is configured to permit ambidextrous operation of the steering mechanism without a user switching from one side of the handle assembly to an opposite side of the handle assembly.

In addition, or alternatively to any example disclosed herein, the handle assembly comprises a torque indicator configured to visually and non-visually convey a rotational position of the handle assembly around a central longitudinal axis of the handle assembly relative to a neutral position.

In addition, or alternatively to any example disclosed herein, the torque indicator comprises a bumped-out region of the handle shell extending longitudinally along the handle shell.

In addition, or alternatively to any example disclosed herein, the longitudinal extension extends proximal of the proximal hub structure.

In addition, or alternatively to any example disclosed herein, and in a second example, a steerable catheter may comprise an elongate sheath extending distally from a handle assembly. The handle assembly may comprise a handle shell including a hand grip portion, a steering mechanism including a knob that is rotatable relative to the handle shell, a longitudinal extension disposed opposite the hand grip portion relative to the knob, and a second longitudinal extension disposed opposite the hand grip portion relative to the knob. The second longitudinal extension may be disposed opposite the longitudinal extension relative to a central longitudinal axis of the handle assembly. The steering mechanism may be configured to deflect a distal portion of the elongate sheath in at least one direction.

In addition, or alternatively to any example disclosed herein, the longitudinal extension has a first length, and the second longitudinal extension has a second length less than the first length.

In addition, or alternatively to any example disclosed herein, the handle assembly comprises a proximal hub structure comprising a proximal hub disposed proximal of the handle shell, a tubular shaft extending distally from the proximal hub within the handle shell, and a valve disposed proximal of the proximal hub.

In addition, or alternatively to any example disclosed herein, the longitudinal extension extends proximal of the valve.

In addition, or alternatively to any example disclosed herein, the longitudinal extension and the second longitudinal extension are fixedly secured to the proximal hub.

In addition, or alternatively to any example disclosed herein, and in a third example, a steerable catheter may comprise an elongate sheath extending distally from a handle assembly. The handle assembly may comprise a handle shell including a hand grip portion, a steering mechanism including a knob that is rotatable relative to the handle shell, and a longitudinal extension disposed opposite the hand grip portion relative to the knob. The handle assembly may be configured to permit concomitant medical procedures requiring both left-handed steering of the elongate sheath and right-handed steering of the elongate sheath.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a steerable catheter;

FIG. 2 illustrates selected aspects of the steerable catheter;

FIG. 3 illustrates selected aspects of the steerable catheter;

FIG. 3A is a cross-sectional view of FIG. 3 taken along line 3A-3A;

FIGS. 4-9 illustrate selected aspects of the steerable catheter;

FIGS. 10-11 illustrate selected aspects of an alternative configuration of the steerable catheter; and

FIG. 12 illustrates selected aspects of an alternative configuration of the steerable catheter.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

In some medical procedures, delivery and/or access sheaths may be routed percutaneously into a body cavity, lumen, and/or treatment site. Navigation through patient vasculature and/or organs may include steering through tortuous anatomy and/or directing a distal end of the delivery and/or access sheath into a body cavity, lumen, and/or treatment site. Examples of steerable catheters suitable for use in medical procedures, such as but not limited to left atrial appendage closure, aortic valve replacement, mitral valve replacement, septal defect repair, atrial fibrillation ablation procedures, etc., are described herein. Existing medical devices may have certain advantages and/or disadvantages.

In some situations, multiple procedures may be performed together (e.g., in parallel or serially). In some situations, different delivery and/or access sheaths have been used to facilitate different handedness of the procedures (e.g., based on user training, preference, etc.). Generally, the different handedness of the procedures requires a user to stand on different or opposite sides of the sheath and/or to grip the handle with either hand depending on procedural requirements. In some procedures, the user may be unable to switch sides of the catheter due to space limitations, equipment and/or room setup, etc. Additionally, where possible, it may be advantageous to use the same delivery and/or access sheath for each procedure – sometimes referred to as concomitant procedures. To address the different handedness of the procedures, a steerable catheter that permits ambidextrous use, without needing to change which side of the catheter that the user is positioned, which provides sufficient grippable surface to enable the user to operate (e.g., steer) the catheter comfortably with either hand, and which does not make the sheath longer (and therefore more difficult to use/handle), is disclosed which may facilitate and/or improve concomitant procedures.

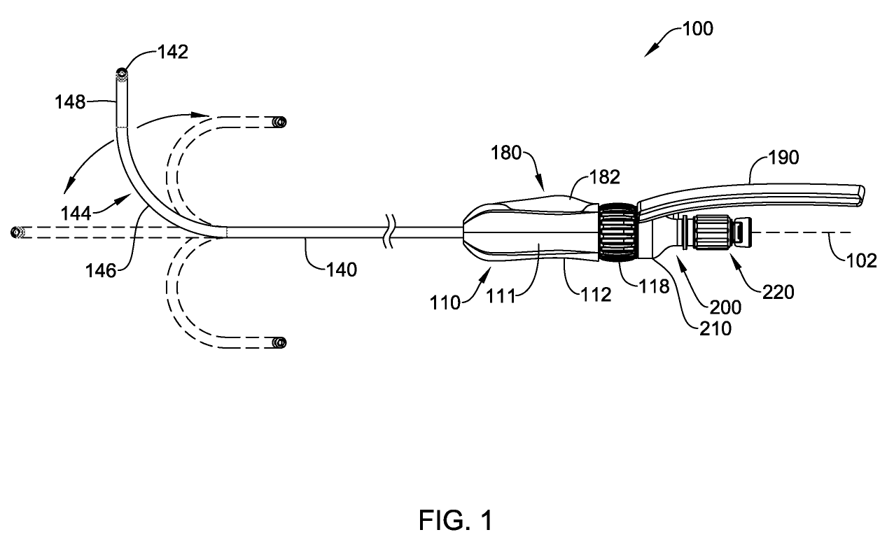

FIG. 1 illustrates selected aspects of a steerable catheter 100. In some embodiments, the steerable catheter 100 may be any one of a variety of catheters, such as an intravascular catheter. Examples of intravascular catheters may include, but are not limited to, balloon catheters, atherectomy catheters, device delivery catheters, drug delivery catheters, diagnostic catheters, and guide catheters. In some embodiments, the steerable catheter 100 may take the form of other suitable guiding, diagnosing, or treating devices (including endoscopic instruments, laparoscopic instruments, etc., and the like) and it may be suitable for use at various locations and/or body lumens within a patient. While the steerable catheter 100 is illustrated and described in one example configuration as a bi-directional steerable catheter, unidirectional, onmidirectional, and/or other types of steerable catheters may also benefit from aspects of the disclosure and therefore the steerable catheter 100 shall not be limited to a bi-directional configuration unless explicitly called out or recited as such.

The steerable catheter 100 may comprise a handle assembly 110 and an elongate sheath 140 extending distally from the handle assembly 110. The steerable catheter 100 and/or the handle assembly 110 may have and/or may extend along a central longitudinal axis 102. In some embodiments, the steerable catheter 100 and/or the handle assembly 110 may include a guidewire port, a fluid flush port, an imaging access port, a Luer connector, and/or other suitable ports, access points, or functional features.

In some embodiments, the elongate sheath 140 may extend into and/or through the handle assembly 110. The handle assembly 110 may comprise a handle housing and/or a handle shell 112. In some embodiments, the elongate sheath 140 may extend into and/or through a distal opening in the handle housing. In some embodiments, the elongate sheath 140 may extend along the central longitudinal axis 102. In some embodiments, the elongate sheath 140 may extend parallel to and/or with the central longitudinal axis 102 within and/or through the handle assembly 110. In some embodiments, the elongate sheath 140 may extend coaxially with the central longitudinal axis 102 within and/or through the handle assembly 110. Other configurations are also contemplated.

In some embodiments, the elongate sheath 140 may be axially secured relative to the handle housing. In some embodiments, a proximal portion of the elongate sheath 140 may include a key element configured to non-rotatably engage one or more lock elements fixedly attached to an inner surface of the handle housing. In some embodiments, the handle housing may include one or more lock elements fixedly attached to and/or monolithically formed with the inner surface of the handle housing. In some embodiments, the one or more lock elements may be formed as ribs or other structural support members configured to increase the rigidity of the handle housing and permit torque transfer between the handle housing and the elongate sheath 140. In some embodiments, the elongate sheath 140 may have a relaxed configuration. The elongate sheath 140 is shown in the relaxed configuration in solid lines in FIG. 1. The elongate sheath 140 may be self-biased toward, and/or in the absence of any outside forces may return to, the relaxed configuration.

In some embodiments, the elongate sheath 140 may include an atraumatic distal tip 142. In some embodiments, the elongate sheath 140 may include a distal portion 144 having a first curve 146 and a second curve 148, such that the elongate sheath 140 has a preset double curve, in the relaxed configuration. In some embodiments, the first curve 146 may be preset to curve upwards, as viewed in FIG. 1. Other configurations are also contemplated. In some embodiments, the second curve 148 may be preset to curve to the left, as viewed proximally to distally along the elongate sheath 140. Other configurations are also contemplated. In some embodiments, the elongate sheath 140 may have only a single curve in the normal or relaxed configuration. In some embodiments, the elongate sheath 140 may be substantially straight in the normal or relaxed configuration. Other configurations, including combinations of those described herein, are also contemplated.

In some embodiments, the handle assembly 110 may comprise a steering mechanism. In at least some embodiments, the steering mechanism may be configured to bend or deflect the distal portion 144 of the elongate sheath 140 in at least one direction. In some embodiments, the steering mechanism may include a knob 118. Selected aspects related to the steering mechanism and the knob 118 are described in more detail below.

In some embodiments, the steering mechanism may be configured to bend or deflect the distal portion 144, and/or the first curve 146 may be configured to bend or deflect, in a first direction, wherein the atraumatic distal tip 142 is bent and/or moved towards and/or closer to the handle assembly 110, toward and/or to a deflected configuration, as shown in phantom in FIG. 1. In some embodiments, the steering mechanism may be configured to bend or deflect the distal portion 144, and/or the first curve 146 may be configured to bend or deflect, in a second direction opposite the first direction, wherein the atraumatic distal tip 142 is bent and/or moved away from and/or farther from the handle assembly 110, toward and/or to a straightened configuration, as shown in phantom in FIG. 1. In some embodiments, the steering mechanism may be configured to bend or deflect the distal portion 144, and/or the first curve 146 may be configured to bend of deflect, further in the second direction beyond the straightened configuration and back toward the handle, as shown in phantom in FIG. 1.

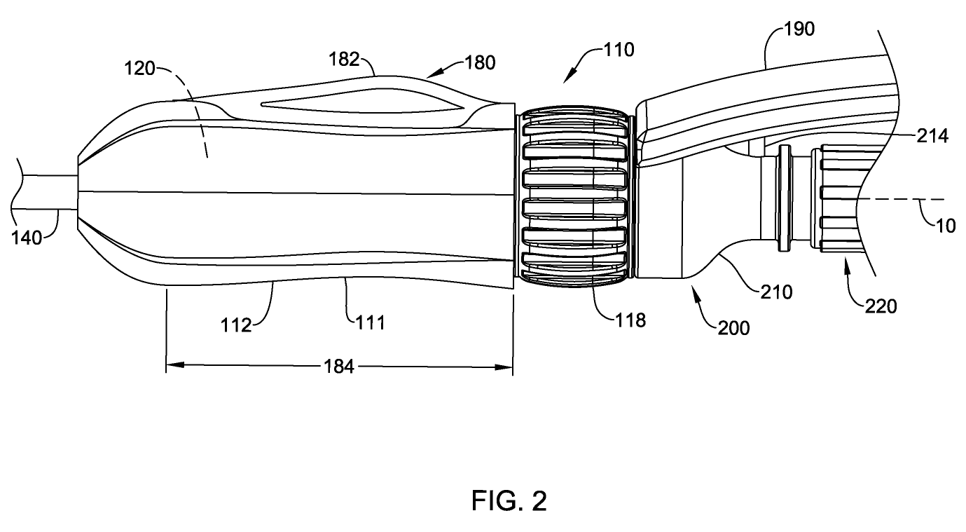

In some embodiments, the handle assembly 110 may comprise a torque indicator 180. In some embodiments, the handle assembly 110 may comprise a longitudinal extension 190. In some embodiments, the handle assembly 110 may comprise a proximal hub structure 200. In some embodiments, the proximal hub structure 200 may comprise a proximal hub 210, a tubular shaft 212 (e.g., FIG. 2), and/or a valve 220. Selected aspects of the torque indicator 180, the longitudinal extension 190, the proximal hub structure 200, and/or elements thereof, are described in more detail below.

FIG. 2 illustrates selected features of the steerable catheter 100 and the handle assembly 110. In some embodiments, the handle shell 112 may comprise a first handle shell (e.g., a right handle shell, a bottom handle shell, etc.) and a second handle shell (e.g., a left handle shell, a top handle shell, etc.). In some embodiments, the handle shell 112 may form a clam shell configuration. In some embodiments, the first handle shell and the second handle shell may be secured together using fasteners, a snap fit, an interference fit, etc. Some suitable but non-limiting materials for the handle assembly 110, the handle housing, the handle shell 112, the first handle shell, the second handle shell, etc., including but not limited to polymeric materials, metallic materials, and/or composite materials, are described below. In some embodiments, the handle assembly 110, the handle housing, the handle shell 112, the first handle shell, the second handle shell may preferably be formed from a polymeric material. In one non-limiting example, the handle assembly 110, the handle housing, the handle shell 112, the first handle shell, the second handle shell may preferably be formed from acrylonitrile butadiene styrene (ABS). Other configurations and/or materials are also contemplated.

In some embodiments, the handle assembly 110 may comprise a steering mechanism. In some embodiments, the steering mechanism may include an axial translation mechanism 120 at least partially disposed within the handle assembly 110 and/or the handle shell 112 of the steerable catheter 100.

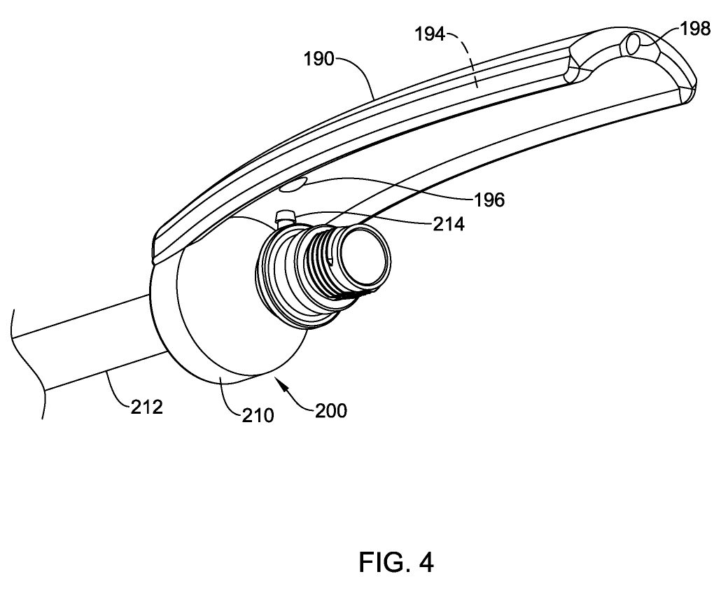

In some embodiments, the proximal hub structure 200 may comprise a proximal hub 210 disposed proximal of the handle shell 112. In some embodiments, the proximal hub structure 200 may comprise a tubular shaft 212 (e.g., FIG. 4) extending distally from the proximal hub 210 within the handle shell 112. In some embodiments, the proximal hub 210 may comprise a side port 214 (for reference, only a portion of the side port 214 is shown in FIG. 2). In some embodiments, the proximal hub structure 200 may comprise a valve 220 disposed proximal of the proximal hub 210 and/or the handle shell 112. In some embodiments, the valve 220 may comprise a check valve, a duck-bill valve, a crosscut valve, a Tuohy valve, or other type of valve, including combinations thereof. In some embodiments, the tubular shaft 212 of the proximal hub structure 200 may extend within and/or through the axial translation mechanism 120. In some embodiments, the elongate sheath 140 may extend within and/or through the axial translation mechanism 120 and/or the tubular shaft 212. In some embodiments, the elongate sheath 140 may be disposed coaxial with the axial translation mechanism 120 and/or the tubular shaft 212.

In some embodiments, the steering mechanism may include a knob 118, wherein the knob 118 is rotatable, and/or is configured to rotate, relative to the handle housing and/or the handle shell 112. In some embodiments, the knob 118 may be configured to rotate and/or actuate the axial translation mechanism 120 within the handle housing and/or the handle shell 112. In some embodiments, the knob 118 may be fixedly attached to the axial translation mechanism 120 outside of the handle housing and/or the handle shell 112. In some embodiments, the knob 118 may be monolithically formed with the axial translation mechanism 120. In some embodiments, the knob 118 may be configured to rotate around and/or relative to the elongate sheath 140. In some embodiments, rotation of the knob 118 relative to the handle housing and/or the handle shell 112 causes rotation and/or actuation of the axial translation mechanism 120 relative to the handle housing and/or the handle shell 112. Some suitable but non-limiting materials for the knob 118, including but not limited to polymeric materials, metallic materials, and/or composite materials, are described below. In some embodiments, the knob 118 may preferably be formed from a polymeric material. In one non-limiting example, the knob 118 may preferably be formed from acrylonitrile butadiene styrene (ABS). Other configurations and/or materials are also contemplated.

In some embodiments, the knob 118 may be axially offset from the handle housing and/or the handle shell 112. In some embodiments, the knob 118 may be proximally offset from the handle housing and/or the handle shell 112. Other configurations are also contemplated. In some embodiments, the knob 118 may be axially spaced apart from the axial translation mechanism 120.

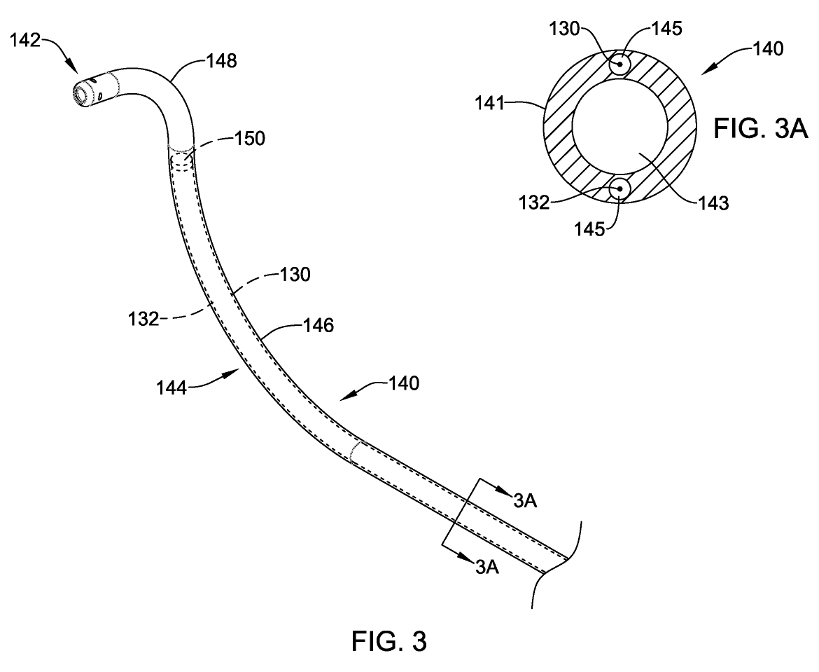

In some embodiments, the steering mechanism may comprise the axial translation mechanism 120 operatively coupled to a first steering wire 130 (e.g., FIG. 3) and a second steering wire 132 (e.g., FIG. 3). The first steering wire 130 and the second steering wire 132 may extend within the handle assembly 110 and/or the handle shell 112 to the elongate sheath 140. In at least some embodiments, the first steering wire 130 and the second steering wire 132 may extend within the elongate sheath 140 from the handle assembly 110 and/or the handle shell 112. In some embodiments, the first steering wire 130 and the second steering wire 132 may be configured to cooperate with the axial translation mechanism 120 to bend and/or deflect the distal portion 144 of the elongate sheath 140 in at least one direction. In some embodiments, the first steering wire 130 may be configured to cooperate with the axial translation mechanism 120 to bend and/or deflect the distal portion 144 of the elongate sheath 140 in the first direction. In some embodiments, the second steering wire 132 may be configured to cooperate with the axial translation mechanism 120 to bend and/or deflect the distal portion 144 of the elongate sheath 140 in the second direction. In some embodiments, the steerable catheter 100 may be a bi-directional steerable catheter. Other configurations are also contemplated.

In one non-limiting example, a first carriage member may be coupled to the first steering wire 130. In some embodiments, the first carriage member may be operatively engaged with the axial translation mechanism 120. In some embodiments, a second carriage member may be coupled to the second steering wire 132. In some embodiments, the second carriage member may be operatively engaged with the axial translation mechanism 120. In some embodiments, the handle housing and/or the handle shell 112 may comprise one or more slide features configured to guide and/or constrain the first carriage member and the second carriage member. In some embodiments, the one or more slide features may permit axial sliding movement of the first carriage member and the second carriage member within the handle housing and/or the handle shell 112. In some embodiments, the one or more slide features may limit and/or constrain the first carriage member and the second carriage member to axial sliding movement within the handle housing and/or the handle shell 112.

In some embodiments, the first carriage member may comprise a concave shape facing radially inward and/or toward the tubular shaft 212. In some embodiments, the first carriage member may comprise a first ear extending away from the concave shape of the first carriage member. The first ear may be configured to receive and/or engage with the first steering wire 130. In some embodiments, the first steering wire 130 may pass through an aperture formed in the first ear. In some embodiments, the first steering wire 130 may comprise a first wire lug disposed proximal of the first ear, wherein the first wire lug is configured to engage with the first ear and is unable to pass through the aperture formed in the first ear. In some embodiments, the first wire lug may be configured to couple the first steering wire 130 to the first carriage member and/or the first ear.

In some embodiments, the second carriage member may comprise a concave shape facing radially inward and/or toward the tubular shaft 212. In some embodiments, the second carriage member may comprise a second ear extending away from the concave shape of the second carriage member. The second ear may be configured to receive and/or engage with the second steering wire 132. In some embodiments, the second steering wire 132 may pass through an aperture formed in the second ear. In some embodiments, the second steering wire 132 may comprise a second wire lug disposed proximal of the second ear, wherein the second wire lug is configured to engage with the second ear and is unable to pass through the aperture formed in the second ear. In some embodiments, the second wire lug may be configured to couple the second steering wire 132 to the second carriage member and/or the second ear. Other configurations and/or examples for the axial translation mechanism 120 and/or aspects thereof are also contemplated.

Turning now to FIG. 3, which illustrates selected aspects of the example steerable catheter, and FIG. 3A, which is a cross-sectional view of FIG. 3 taken along the line 3A-3A, the elongate sheath 140 may include a wall 141 defining a central lumen 143 extending from a proximal end to the soft and/or atraumatic distal tip 142 along the central longitudinal axis of the elongate sheath 140. In some embodiments, the central lumen 143 may be coaxial with the central longitudinal axis of the elongate sheath 140. In some embodiments, the central lumen 143 may be a guidewire lumen. In some embodiments, the central lumen 143 may be a device lumen used to deliver a medical device or implant. In some embodiments, the central lumen 143 may have multiple uses. The elongate sheath 140 may include a plurality of steering wire lumens 145 extending and/or disposed within the wall 141. In some embodiments, the plurality of steering wire lumens 145 may include a first steering wire lumen and a second steering wire lumen. In some embodiments, the plurality of steering wire lumens 145 may include more than two steering wire lumens. In some embodiments, the plurality of steering wire lumens 145 may be oriented substantially parallel to the central lumen 143 and/or the central longitudinal axis of the elongate sheath 140. In some embodiments, the plurality of steering wire lumens 145 may be disposed opposite each other and/or on opposite sides of the elongate sheath 140 relative to the central lumen 143 and/or the central longitudinal axis of the elongate sheath 140. Other configurations are also contemplated.

In some embodiments, the plurality of steering wire lumens 145 may have a circular cross-sectional shape, as shown in FIG. 3A. However, the cross-sectional shape shown in FIG. 3A is merely exemplary and is not intended to be limiting. In some embodiments, the plurality of steering wire lumens 145 may have other cross-sectional shapes. For example, in some embodiments, the plurality of steering wire lumens 145 may have a rectangular cross-sectional shape, an ovoid cross-sectional shape, a square cross-sectional shape, a polygonal cross-sectional shape, etc. In some embodiments, the plurality of steering wire lumens 145 may have a cross-sectional shape that is regular and/or symmetrical. In some embodiments, the plurality of steering wire lumens 145 may have a cross-sectional shape that is irregular and/or asymmetrical. Other configurations are also contemplated.

In some embodiments, a distal pull ring 150 may be disposed within the distal portion 144 of the elongate sheath 140. In some embodiments, the distal pull ring 150 may be disposed proximal to the second curve 148 and/or the atraumatic distal tip 142. In some embodiments, the distal pull ring 150 may be disposed proximate a distal end of the first curve 146. In some embodiments, the distal pull ring 150 may be embedded within the wall 141 of the elongate sheath 140. In some embodiments, the distal pull ring 150 may be secured, bonded, and/or fixedly attached to an inner surface of the wall 141 of the elongate sheath 140. Other configurations are also contemplated. Some suitable but non-limiting materials for the distal pull ring 150 are described below.

The first steering wire 130 and the second steering wire 132 may each be slidably disposed within the plurality of steering wire lumens 145. In one example, the first steering wire 130 may be slidably disposed within a first steering wire lumen and the second steering wire 132 may be disposed within a second steering wire lumen. The first steering wire 130 and the second steering wire 132 may be fixedly attached (e.g., bonded, welded, etc.) to the distal pull ring 150. For example, a distal end of the first steering wire 130 may be fixedly attached to the distal pull ring 150 and a distal end of the second steering wire 132 may be fixedly attached to the distal pull ring 150 at a position opposite the distal end of the first steering wire 130 relative to the central longitudinal axis of the elongate sheath 140. Some suitable but non-limiting materials for the first steering wire 130 and the second steering wire 132 are described below.

In some embodiments, the elongate sheath 140 may be sized in accordance with its intended use. For example, the elongate sheath 140 can have a length that is in the range of about 50 to about 200 centimeters (19.69 to 78.74 inches), about 75 to about 175 centimeters (29.53 to 68.90 inches), or about 100 to about 150 centimeters (39.37 to 59.06 inches). Other lengths are also contemplated. It is further contemplated that the outer diameter and/or the inner diameter of the elongate sheath 140 may vary based on the use or application. In some examples, the outer diameter of the elongate sheath 140 may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm (or 18 French), about 6.5 mm, about 7 mm (or 21 French), about 8 mm (or 24 French), or other suitable sizes. In some embodiments, the outer diameter of the elongate sheath 140 may be a maximum of 5.66 mm (17 French), and is preferably smaller than 5.66 mm (17 French). Other configurations are also contemplated. Some suitable but non-limiting materials for the elongate sheath 140, which may include polymeric materials, metallic materials, composite materials, etc., are described below.

The first steering wire 130 may extend distally from the axial translation mechanism 120 to the distal pull ring 150 disposed within the elongate sheath 140. The second steering wire 132 may extend distally from the axial translation mechanism 120 to the distal pull ring 150 disposed within the elongate sheath 140. The first steering wire 130 may extend distally within the elongate sheath 140 (e.g., within the first steering wire lumen of the plurality of steering wire lumens 145) from the handle housing and/or within the handle shell 112 to the distal pull ring 150. The second steering wire 132 may extend distally within the elongate sheath 140 (e.g., within the second steering wire lumen of the plurality of steering wire lumens 145) from the handle housing and/or the handle shell 112 to the distal pull ring 150. The second steering wire 132 may be disposed on an opposite side of the elongate sheath 140 from the first steering wire 130 relative to the central longitudinal axis of the elongate sheath 140.

In some embodiments, the steering mechanism and/or the axial translation mechanism 120 may be configured to shift the elongate sheath 140 extending distally from the handle assembly 110 between the relaxed configuration, the deflected configuration, and the straightened configuration.

In some embodiments, rotation of the knob 118 may be configured to actuate the axial translation mechanism 120. In some embodiments, rotation of the knob 118 may be configured to axially translate the first carriage member and the second carriage member within the handle housing and/or the handle shell 112. In some embodiments, rotation of the knob 118 may be configured to axially translate the first carriage member and the second carriage member in opposite directions within the handle housing and/or the handle shell 112. In some embodiments, actuation of the axial translation mechanism 120 may be configured to selectively apply tension to the first steering wire 130 and/or the second steering wire 132 to shift the elongate sheath 140 between the relaxed configuration, the deflected configuration, and the straightened configuration. In some embodiments, the first carriage member and the second carriage member may be configured to axially pass by each other as they axially translate within the handle housing and/or the handle shell 112. Other configurations are also contemplated.

In some embodiments, actuation of the axial translation mechanism 120 may be configured to selectively apply tension to the first steering wire 130 and/or the second steering wire 132 to shift the elongate sheath 140 between the relaxed configuration, the deflected configuration, and the straightened configuration. In some embodiments, actuation of the axial translation mechanism 120 may be configured to selectively apply tension to the first steering wire 130 and/or the second steering wire 132 to bend and/or deflect the distal portion 144 and/or the first curve 146 of the elongate sheath 140. As discussed herein, the axial translation mechanism 120 may be configured to shift the elongate sheath 140 between the relaxed configuration, the deflected configuration wherein the distal portion 144 of the elongate sheath 140 is bent in the first direction from the relaxed configuration, and the straightened configuration wherein the distal portion 144 of the elongate sheath 140 is bent in the second direction from the relaxed configuration.

The first steering wire 130 may be configured to engage the axial translation mechanism 120 to bend and/or deflect the distal portion 144 and/or the first curve 146 of the elongate sheath 140 in the first direction toward the handle assembly 110 and/or the handle housing, toward and/or to the deflected configuration. The second steering wire 132 may be configured to engage the axial translation mechanism 120 to bend and/or deflect the distal portion 144 and/or the first curve 146 of the elongate sheath 140 in the second direction opposite the first direction and away from the handle assembly 110 and/or the handle housing, toward and/or to the straightened configuration. The tension applied by the axial translation mechanism 120 may be sufficient to overcome the self-bias of the elongate sheath 140 toward the normal or relaxed configuration and bend and/or deflect the distal portion 144 and/or the first curve 146 of the elongate sheath 140 in the first direction and/or the second direction.

In some embodiments, clockwise rotation of the knob 118 may be configured to and/or may cause proximal movement and/or proximal axial translation of the first carriage member. Proximal movement and/or proximal axial translation of the first carriage member may apply tension to the first steering wire 130, thereby shifting the elongate sheath 140 toward and/or to the deflected configuration. In some embodiments, clockwise rotation of the knob 118 may be configured to and/or may cause distal movement and/or distal axial translation of the second carriage member. Distal movement and/or distal axial translation of the second carriage member may release stored tension within the second steering wire 132 and/or may permit the second steering wire 132 to translate distally within the elongate sheath 140 such that the distal pull ring 150 may be pulled by the first steering wire 130.

In some embodiments, counterclockwise rotation of the knob 118 may be configured to and/or may cause proximal movement and/or proximal axial translation of the second carriage member. Proximal movement and/or proximal axial translation of the second carriage member may apply tension to the second steering wire 132, thereby shifting the elongate sheath 140 toward and/or to the straightened configuration. In some embodiments, clockwise rotation of the knob 118 may be configured to and/or may cause distal movement and/or distal axial translation of the first carriage member. Distal movement and/or distal axial translation of the first carriage member may release stored tension within the first steering wire 130 and/or may permit the first steering wire 130 to translate distally within the elongate sheath 140 such that the distal pull ring 150 may be pulled by the second steering wire 132.

Returning to FIGS. 1-2, in some embodiments, the handle assembly 110 and/or the handle shell 112 may comprise a torque indicator 180 configured to visually and non-visually convey, provide, and/or communicate a rotational position of the handle assembly 110 and/or the handle shell 112 around the central longitudinal axis 102 of the handle assembly 110 relative to a neutral position of the handle assembly 110 and/or the handle shell 112 to a user. In at least some embodiments, the torque indicator 180 may be configured to convey, provide, and/or communicate the rotational position of the handle assembly 110 and/or the handle shell 112 around the central longitudinal axis 102 of the handle assembly 110 relative to the neutral position of the handle assembly 110 and/or the handle shell 112 to the user without the user needing to look at the handle assembly 110 and/or the handle shell 112 (e.g., without the user needing to look away from a fluoroscopy screen, for example). In some embodiments, the torque indicator 180 may be and/or may function as a curve indicator configured to visually and non-visually convey, provide, and/or communicate a position of the distal portion 144 and/or the first curve 146 of the elongate shaft 140 relative to a neutral position of the distal portion 144 and/or the first curve 146 of the elongate shaft 140.

In some embodiments, the torque indicator 180 may improve the user interface during a procedure with the steerable catheter 100. In some procedures, the steerable catheter 100 must be able to be torqued and/or deflected using only one hand. In some embodiments, the torque indicator 180 may improve control over the steerable catheter 100 during the procedure. In some embodiments, the torque indicator 180 may improve ease of use and/or intuitiveness of the steerable catheter 100.

In some embodiments, the torque indicator 180 may comprise a tactile feature formed on a hand grip portion 111 of the handle housing and/or the handle shell 112. As such, the torque indicator 180 and/or the tactile feature may be felt by the user’s hand as the user grips the hand grip portion 111 of the handle housing and/or the handle shell 112, thereby negating a need to look at the handle assembly 110, the handle housing, and/or the handle shell 112 to determine an orientation and/or the rotational position thereof relative to the neutral position. In some embodiments, the torque indicator 180 and/or the tactile feature may comprise a bumped-out region 182 of the handle housing and/or the handle shell 112 extending longitudinally along the handle housing and/or the handle shell 112. In some embodiments, the bumped-out region 182 of the handle housing and/or the handle shell 112 may be disposed between a proximal end of the hand grip portion 111 of the handle housing and/or the handle shell 112 and a distal end of the hand grip portion 111 of the handle housing and/or the handle shell 112. In some embodiments, the torque indicator 180 and/or the tactile feature may extend along more than 50% of a length 184 (e.g., FIG. 2) of the hand grip portion 111 of the handle housing and/or the handle shell 112. In some embodiments, the torque indicator 180 and/or the tactile feature may extend along less than 100% of the length 184 (e.g., FIG. 2) of the hand grip portion 111 of the handle housing and/or the handle shell 112. In some alternative configurations, the torque indicator 180 and/or the tactile feature may extend along 100% of the length 184 (e.g., FIG. 2) (e.g., an entire length) of the hand grip portion 111 of the handle housing and/or the handle shell 112. Other configurations are also contemplated.

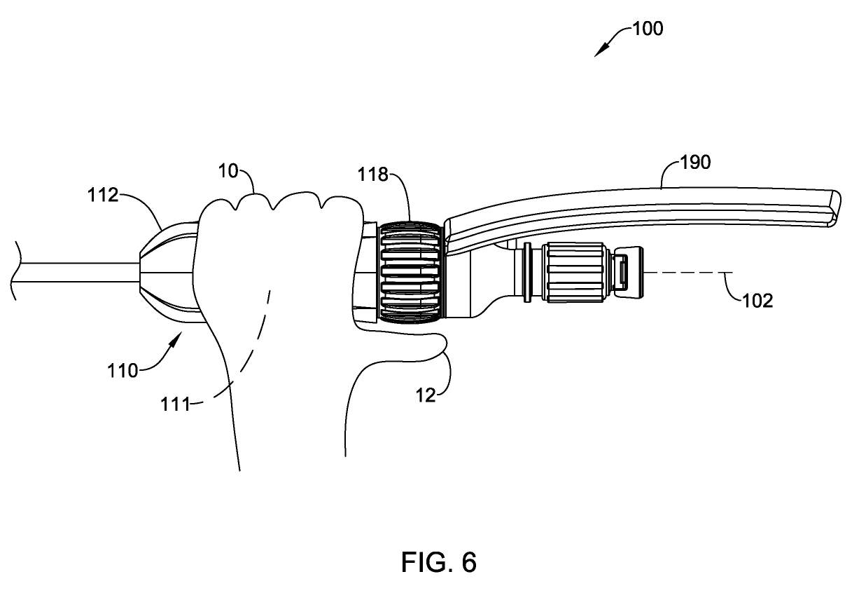

In at least some embodiments, the handle assembly 110 may be adapted and/or configured to permit and/or to facilitate ambidextrous operation of the steerable catheter 100, the steering mechanism, and/or the knob 118 (e.g., the user may be able to switch between left-handed or right-handed operation) without the user switching from one side of the handle assembly 110 to an opposite side of the handle assembly 110. For example, in FIG. 1, the user could be positioned facing the handle assembly 110 from a front side (e.g., facing into the page and positioned in front of the handle assembly 110 as shown) or from a back side (e.g., facing out from the page and positioned behind the handle assembly 110 as shown), and being able to ambidextrously operate the steerable catheter 100, the steering mechanism, and/or the knob 118. In some embodiments, the hand grip portion 111 of the handle housing and/or the handle shell 112 may be configured to receive and/or be gripped by a user’s left hand or a user’s right hand (as seen in FIGS. 6-9 and discussed further below), depending on which side of the handle assembly 110 the user is positioned on or which hand is needed to steer, bend, or deflect the distal portion 144 of the elongate shaft 140 for a particular procedure.

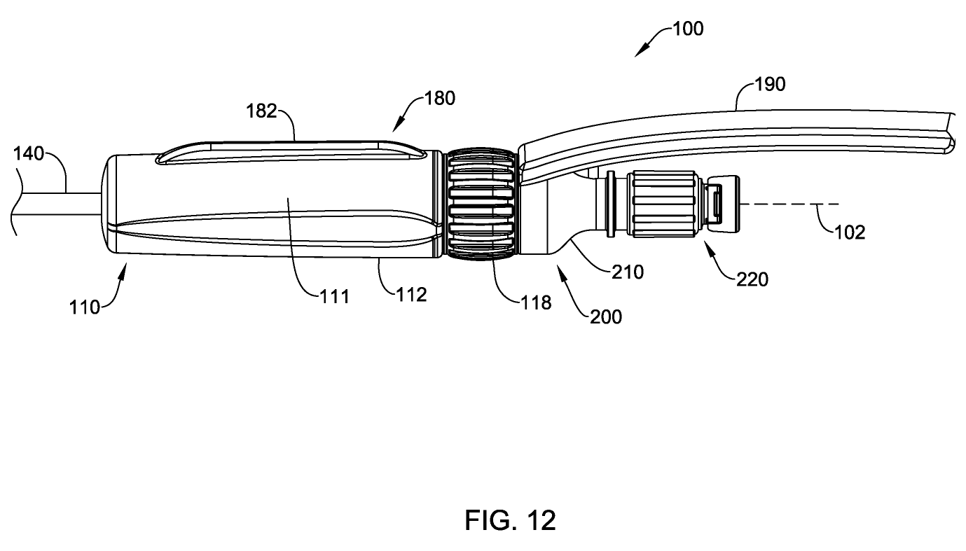

In some embodiments, the torque indicator 180, the tactile feature, and/or the bumped-out region 182 may be curved, as seen in the example configuration of FIG. 1. In some embodiments, the torque indicator 180, the tactile feature, and/or the bumped-out region 182 may be configured to correspond to a user’s natural grip and/or hand shape. In some embodiments, the torque indicator 180, the tactile feature, and/or the bumped-out region 182 may form a longitudinal rib, as seen in the example and/or alternative configuration of FIG. 12. Other aspects of the example and/or alternative configuration of FIG. 12 may be substantially the same as that of earlier figures. In some embodiments, the longitudinal rib may be oriented substantially parallel to the central longitudinal axis 102 of the handle assembly 110. In some embodiments, the longitudinal rib may have substantially parallel sides extending outward from the handle housing and/or the handle shell 112. Other configurations are also contemplated.

As discussed above, in some embodiments, the handle assembly 110 may comprise a longitudinal extension 190. In some embodiments, the longitudinal extension 190 may be offset from the central longitudinal axis 102 of the handle assembly 110. In some embodiments, the longitudinal extension 190 may be disposed opposite the hand grip portion 111 of the handle shell 112 relative to the knob 118. In some embodiments, the longitudinal extension 190 may extend proximal of the proximal hub structure 200 and/or the valve 220.

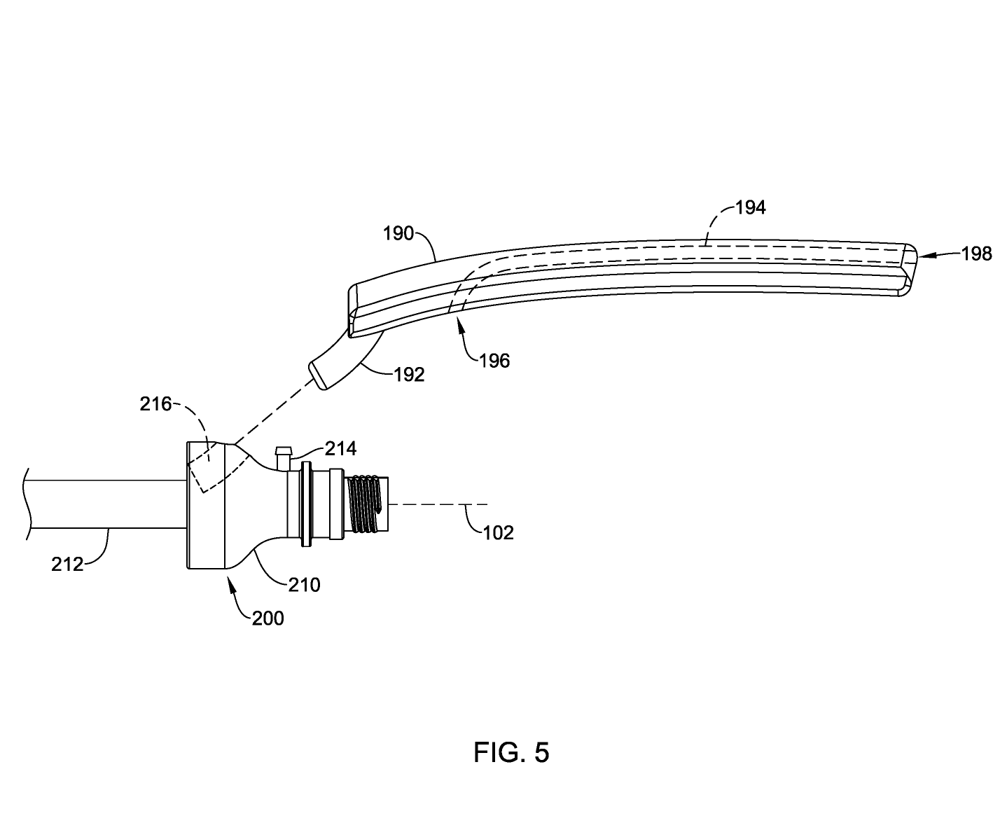

Turning now to FIGS. 4-5, selected aspects of the proximal hub structure 200 are shown in more detail. In some embodiments, the proximal hub structure 200 may comprise a proximal hub 210 and a tubular shaft 212 extending distally from the proximal hub 210. In some embodiments, the tubular shaft 212 may be fixedly attached to the proximal hub 210. In some embodiments, the tubular shaft 212 may be monolithically formed with the proximal hub 210. In some embodiments, the proximal hub 210 may comprise a side port 214.

In some embodiments, the longitudinal extension 190 may have an arcuate shape around, and/or a concave shape opening toward, the central longitudinal axis 102 of the handle assembly 110 (e.g., FIGS. 1-2). In some embodiments, the arcuate shape and/or the concave shape may permit easier and/or more comfortable gripping of the longitudinal extension 190 by the user’s hand and/or fingers. In some embodiments, the arcuate shape and/or the concave shape may also maintain access to the proximal hub 210 and/or the valve 220 without enlarging and/or extending the handle shell 112.

In some embodiments, the longitudinal extension 190 may be coupled directly to the proximal hub 210. In some embodiments, the longitudinal extension 190 may comprise a projection 192 extending therefrom. In some embodiments, the proximal hub 210 may comprise a receptacle 216 that opens radially outward. In some embodiments, the receptacle 216 may open radially outward in a proximal direction. In some embodiments, the projection 192 of the longitudinal extension 190 may be configured to matingly engage with the receptacle 216 of the proximal hub 210. In some embodiments, the longitudinal extension 190 may be detachable from the proximal hub 210. In some embodiments, the projection 192 of the longitudinal extension 190 may be configured to engage with the receptacle 216 of the proximal hub 210 via a friction fit, a press fit, an interference fit, mechanical attachment, etc. In some embodiments, the longitudinal extension 190 may be fixedly secured and/or fixedly attached to the proximal hub 210. In some embodiments, the longitudinal extension 190 may be non-removably engaged with the proximal hub 210. In one non-limiting example, the projection 192 of the longitudinal extension 190 may be adhesively bonded within the receptacle 216 of the proximal hub 210. Other configurations are also contemplated. In some embodiments, the longitudinal extension 190 may be monolithically formed with the proximal hub 210.

In some embodiments, the longitudinal extension 190 may comprise a lumen 194 extending longitudinally therein. In some embodiments, the lumen 194 may extend from a distal opening 196 to a proximal opening 198. In at least some embodiments, the distal opening 196 may be disposed proximal of a distal end of the longitudinal extension 190. In some embodiments, the lumen 194 of the longitudinal extension 190 may open toward the central longitudinal axis 102 of the handle assembly 110 (e.g., FIGS. 1-2) proximate the side port 214 of the proximal hub 210. In some embodiments, the lumen 194 of the longitudinal extension 190 may be sized and/or configured to permit a tube to be passed therethrough such that a distal end of the tube may be fluidly coupled to the side port 214 without kinking the tube and/or while protecting the tube. In some alternative configurations, the lumen 194 of the longitudinal extension 190 may be fluidly coupled directly to the side port 214. For example, in some embodiments where the longitudinal extension 190 is monolithically formed with the proximal hub 210, the side port 214 may extend completely to the longitudinal extension 190 such that there is no gap between the side port 214 and the longitudinal extension 190 (e.g., the proximal hub 210 may be in direct fluid communication with the lumen 194 of the longitudinal extension 190). Other configurations are also contemplated.

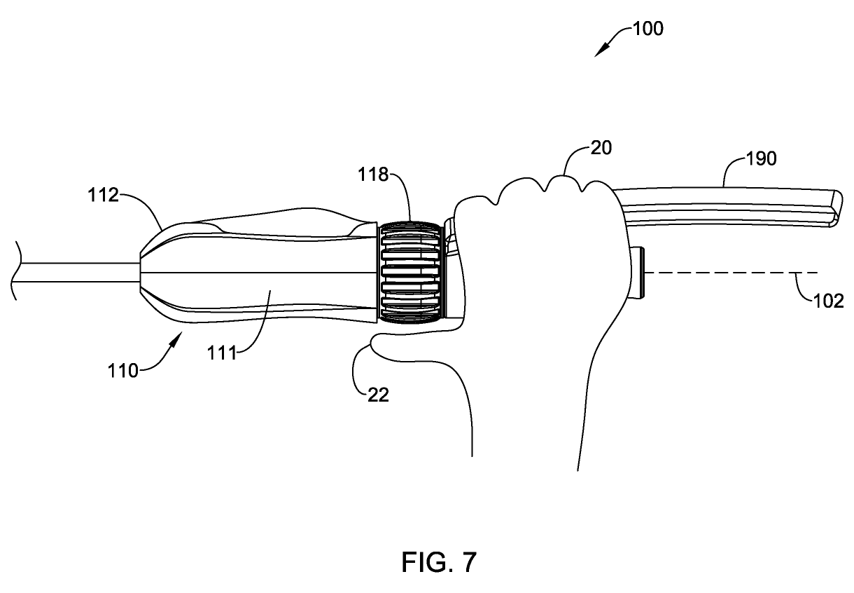

In some embodiments, the knob 118 may be rotatable by a first hand 10 of a user disposed on the hand grip portion 111 of the handle shell 112, as seen in FIG. 6, and/or by a second hand 20 of the user disposed on the longitudinal extension 190, as seen in FIG. 7. In some embodiments, the first hand 10 may be a left hand of the user, as seen in FIG. 6, and the second hand 20 may be a right hand of the user, as seen in FIG. 7. Accordingly, a first thumb 12 (e.g., a left thumb) of the user may be positioned to rotate the knob 118 when left-handed steering is desired, as seen in FIG. 6, and a second thumb 22 (e.g., a right thumb) of the user may be positioned to rotate the knob 118 when right-handed steering is desired, as seen in FIG. 7. This arrangement permits ambidextrous operation of the steering mechanism without a user switching from one side of the handle assembly 110 to an opposite side of the handle assembly 110.

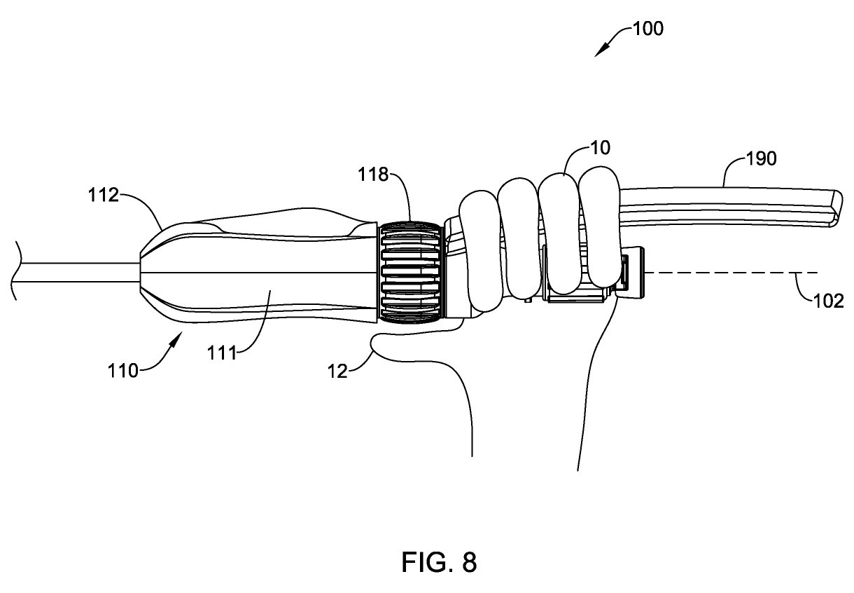

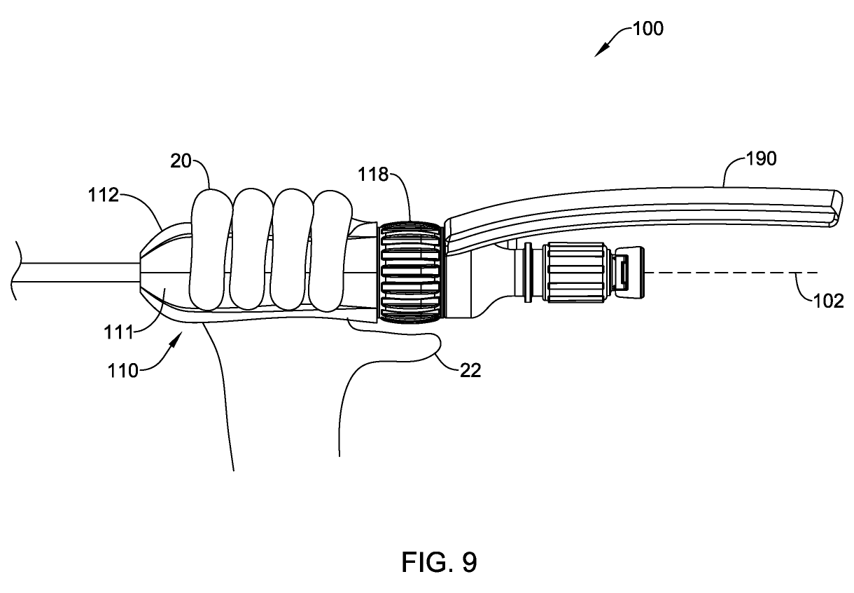

Alternatively, if the user was positioned on the opposite side of the handle assembly 110 compared to the positioning shown in FIGS. 6-7, the first hand 10 (e.g., the left hand) of the user may be disposed on the longitudinal extension 190 such that the first thumb 12 (e.g., the left thumb) is positioned to operate and/or rotate the knob 118 when left-handed steering is desired, as seen in FIG. 8, and the second hand 20 (e.g., the right hand) of the user may be disposed on the hand grip portion 111 of the handle shell 112 such that the second thumb 22 (e.g., the right thumb) of the user is positioned to rotate the knob 118 when right-handed steering is desired, as seen in FIG. 9. This arrangement also permits ambidextrous operation of the steering mechanism without a user switching from one side of the handle assembly 110 to an opposite side of the handle assembly 110.

In at least some embodiments, the handle assembly 110 may be configured to permit concomitant medical procedures requiring both left-handed steering of the elongate sheath 140 and right-handed steering of the elongate sheath 140. Accordingly, the need for multiple delivery and/or access sheaths may be avoided. In some embodiments, the handle assembly 110 may be configured to reduce the number of hand positions the user uses and/or cycles through during a procedure, which may result in a more efficient workflow. In some embodiments, the handle assembly 110 may be configured to improve user control by reducing the number of switches between different hand positions used during a procedure. Other benefits and/or advantages are also contemplated.

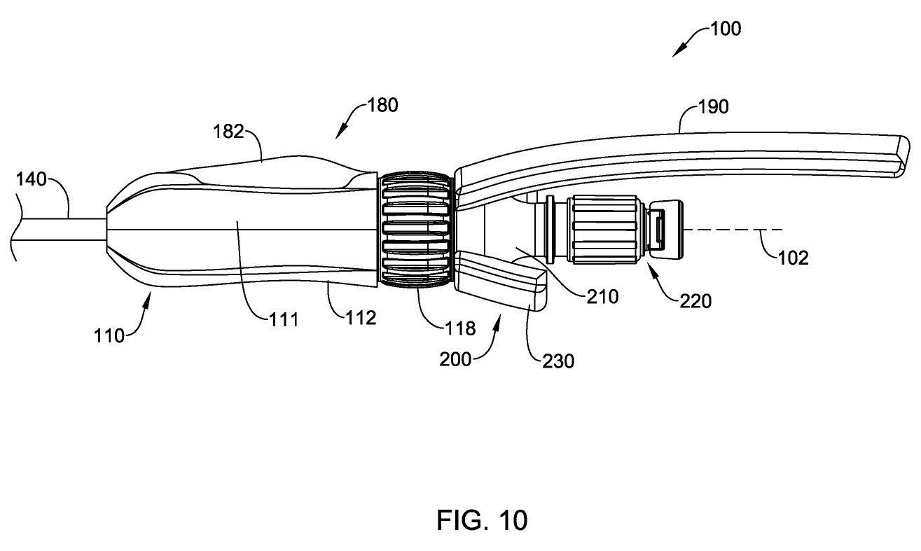

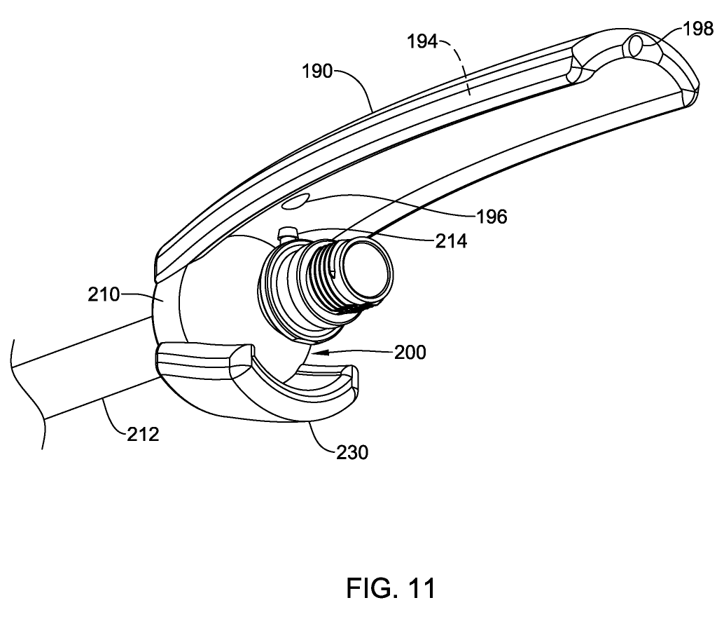

Turning now to FIGS. 10-11, in some embodiments, the handle assembly 110 may comprise a second longitudinal extension 230 disposed opposite the hand grip portion 111 of the handle shell 112 relative to the knob 118. In some embodiments, the second longitudinal extension 230 may be offset from the central longitudinal axis 102 of the handle assembly 110. In some embodiments, the second longitudinal extension 230 may be disposed opposite the longitudinal extension 190 relative to the central longitudinal axis 102 of the handle assembly 110. In at least some embodiments, the proximal hub 210 may extend proximal of the second longitudinal extension 230. In at least some embodiments, the longitudinal extension 190 has a first length, and the second longitudinal extension 230 has a second length less than the first length.

In some embodiments, the second longitudinal extension 230 may have an arcuate shape around, and/or a concave shape opening toward, the central longitudinal axis 102 of the handle assembly 110. In some embodiments, the arcuate shape and/or the concave shape may permit easier and/or more comfortable engagement of the second longitudinal extension 230 by the user’s hand and/or thumb. In some embodiments, the arcuate shape and/or the concave shape may also maintain access to the proximal hub 210 and/or the valve 220 without enlarging and/or extending the handle shell 112.

In some embodiments, the second longitudinal extension 230 may be coupled directly to the proximal hub 210. In some embodiments, the second longitudinal extension 230 may comprise a projection (not shown; may be similar to the projection 192 of the longitudinal extension 190) extending therefrom. In some embodiments, the proximal hub 210 may comprise a second receptacle (not shown; may be similar to the receptacle 216) that opens radially outward. In some embodiments, the second receptacle may open radially outward in a proximal direction. In some embodiments, the second projection of the second longitudinal extension 230 may be configured to matingly engage with the second receptacle of the proximal hub 210. In some embodiments, the second longitudinal extension 230 may be detachable from the proximal hub 210. In some embodiments, the second projection of the second longitudinal extension 230 may be configured to engage with the second receptacle of the proximal hub 210 via a friction fit, a press fit, an interference fit, mechanical attachment, etc. In some embodiments, the second longitudinal extension 230 may be fixedly secured and/or fixedly attached to the proximal hub 210. In some embodiments, the second longitudinal extension 230 may be non-removably engaged with the proximal hub 210. In one non-limiting example, the second projection of the second longitudinal extension 230 may be adhesively bonded within the second receptacle of the proximal hub 210. Other configurations are also contemplated. In some embodiments, the second longitudinal extension 230 may be monolithically formed with the proximal hub 210.

In some embodiments, the steerable catheter 100 having the second longitudinal extension 230 may be useful and/or more comfortable for users with larger hands. The second longitudinal extension 230 may permit the base of the user’s thumb to rest thereon without interfering with other structures and/or movements (e.g., the right thumb may still be able to engage and/or rotate the knob 118 for right-handed steering, similar to FIG. 7). In some embodiments (like in the arrangement and/or positioning of FIG. 6) where left-handed steering is desired, the right hand of the user may grasp around the longitudinal extension 190 and the second longitudinal extension 230 (e.g., the right thumb may rest on, grip, or wrap around the second longitudinal extension 230). Alternatively, if the user was positioned on the opposite side of the handle assembly 110 compared to the positioning shown in FIG. 6, similar to FIG. 8, the base of the user’s left thumb may be configured to rest thereon without interfering with other structures and/or movements (e.g., the left thumb may still be able to engage and/or rotate the knob 118 for left-handed steering). Other configurations and/or benefits are also contemplated. In some embodiments, the second longitudinal extension 230 may provide and/or may increase available surface area for gripping by the user’s hand. In some embodiments, the second longitudinal extension may be configured to interact with the user’s hand to provide countertorque when operating and/or rotating the knob 118.

The materials that can be used for the various components of the steerable catheter (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the handle assembly, the elongate sheath, the steering mechanism, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. A steerable catheter, comprising:
an elongate sheath extending distally from a handle assembly;
wherein the handle assembly comprises:
a handle shell including a hand grip portion;
a steering mechanism including a knob that is rotatable relative to the handle shell; and
a longitudinal extension disposed opposite the hand grip portion relative to the knob;
wherein the steering mechanism is configured to deflect a distal portion of the elongate sheath in at least one direction.

2. The steerable catheter of claim 1, wherein the longitudinal extension is offset from a central longitudinal axis of the handle assembly.

3. The steerable catheter of claim 1, wherein the handle assembly comprises a proximal hub structure comprising a proximal hub disposed proximal of the handle shell and a tubular shaft extending distally from the proximal hub within the handle shell.

4. The steerable catheter of claim 3, wherein the longitudinal extension is coupled directly to the proximal hub.

5. The steerable catheter of claim 4, wherein the longitudinal extension is monolithically formed with the proximal hub.

6. The steerable catheter of claim 4, wherein the longitudinal extension is detachable from the proximal hub.

7. The steerable catheter of claim 6, wherein the proximal hub comprises a receptacle that opens radially outward in a proximal direction and the longitudinal extension comprises a projection configured to matingly engage with the receptacle.

8. The steerable catheter of claim 3, wherein the longitudinal extension comprises a lumen extending longitudinally therein.

9. The steerable catheter of claim 6, wherein the lumen of the longitudinal extension opens toward a central longitudinal axis of the handle assembly proximate a side port of the proximal hub.

10. The steerable catheter of claim 1, wherein the knob is rotatable by a first hand of a user disposed on the hand grip portion of the handle shell and by a second hand of the user disposed on the longitudinal extension.

11. The steerable catheter of claim 1, wherein the handle assembly is configured to permit ambidextrous operation of the steering mechanism without a user switching from one side of the handle assembly to an opposite side of the handle assembly.

12. The steerable catheter of claim 1, wherein the handle assembly comprises a torque indicator configured to visually and non-visually convey a rotational position of the handle assembly around a central longitudinal axis of the handle assembly relative to a neutral position.

13. The steerable catheter of claim 12, wherein the torque indicator comprises a bumped-out region of the handle shell extending longitudinally along the handle shell.

14. The steerable catheter of claim 3, wherein the longitudinal extension extends proximal of the proximal hub structure.

15. A steerable catheter, comprising:
an elongate sheath extending distally from a handle assembly;
wherein the handle assembly comprises:
a handle shell including a hand grip portion;
a steering mechanism including a knob that is rotatable relative to the handle shell;
a longitudinal extension disposed opposite the hand grip portion relative to the knob; and
a second longitudinal extension disposed opposite the hand grip portion relative to the knob;
wherein the second longitudinal extension is disposed opposite the longitudinal extension relative to a central longitudinal axis of the handle assembly;
wherein the steering mechanism is configured to deflect a distal portion of the elongate sheath in at least one direction.

16. The steerable catheter of claim 15, wherein the longitudinal extension has a first length, and the second longitudinal extension has a second length less than the first length.

17. The steerable catheter of claim 15, wherein the handle assembly comprises a proximal hub structure comprising:
a proximal hub disposed proximal of the handle shell;
a tubular shaft extending distally from the proximal hub within the handle shell; and
a valve disposed proximal of the proximal hub.

18. The steerable catheter of claim 17, wherein the longitudinal extension extends proximal of the valve.

19. The steerable catheter of claim 17, wherein the longitudinal extension and the second longitudinal extension are fixedly secured to the proximal hub.

20. A steerable catheter, comprising:
an elongate sheath extending distally from a handle assembly;
wherein the handle assembly comprises:
a handle shell including a hand grip portion;
a steering mechanism including a knob that is rotatable relative to the handle shell; and
a longitudinal extension disposed opposite the hand grip portion relative to the knob;
wherein the handle assembly is configured to permit concomitant medical procedures requiring both left-handed steering of the elongate sheath and right-handed steering of the elongate sheath.
